(19) United States
(12) Patent Application Publication
(10) Pub. No.: US 2025/0205466 A1
(43) Pub. Date: Jun. 26, 2025

(54) Tattoo Machine and Tool

(71) Applicant: FK Irons Inc., Doral, FL (US)

(72) Inventor: Christian Joseph Bonomo, Homestead, FL (US)

(73) Assignee: FK Irons Inc., Doral, FL (US)

(21) Appl. No.: 18/990,452

(22) Filed: Dec. 20, 2024

Related U.S. Application Data
(60) Provisional application No. 63/614,332, filed on Dec. 22, 2023.

(51) Int. Cl. A61M 37/00

(52) U.S. Cl. CPC A61M 37/0076

(57) ABSTRACT

A tattoo machine and tool are disclosed. The tattoo machine includes a housing, a motor assembly, and a tool. The motor assembly includes a motor block, a translator, a bearing, and a cam assembly. The translator includes a translator arm and a translator body. The translator arm can be fixedly attached or detachably secured to a translator body. If the translator arm is detachably secured to the translator body, the tool can be used to adjust the horizontal displacement of the translator arm, which changes the penetration depth of the needle. The tool is used to loosen the detachable connection between the translator arm and the translator body, slide the translator arm to the desired position, tighten the detachable connection, then reassemble the motor assembly.

### CROSS REFERENCE TO RELATED APPLICATIONS

This application claims the priority benefit of Provisional Application No. 63/614,332 filed on Dec. 22, 2023, which is incorporated by reference in its entirety.

### FIELD OF THE APPLICATION

The present application relates to tattoo, permanent makeup and micropigmentation machines.

### BACKGROUND

Tattoo machines, commonly referred to as tattoo pens, are used to apply tattoo ink, permanent makeup and pigments to a person's body and skin. A tattoo pen body is typically cylindrically shaped for ease of the tattoo artist's grip and comfort when tattooing a customer. Tattoo pens with batteries allow for a user to freely orientate the tattoo pen for the best orientation for drawing on what can be uneven surfaces. Tattoo pens can have removable batteries. The tattoo pen provides an artist with optimal functionality and comfort when applying tattoos. Such functionality includes being free to tattoo customers both inside a traditional studio and other locations.

### SUMMARY

A tattoo device comprises a tattoo machine and an tool. The tattoo machine further comprises a tattoo machine housing defining a longitudinal central axis and a motor assembly housed within the tattoo machine housing. The tattoo machine housing further comprises a grip section, a motor housing that detachably couples with the grip section; and a battery housing that detachably couples with the motor housing.

In an embodiment of the tattoo machine, the motor housing further comprises an exterior motor housing and an interior motor carrier. The interior motor carrier is disposed within an inner cavity of the exterior motor housing, wherein the interior motor carrier detachably couples with the exterior motor housing. The tool is carried by the interior motor carrier.

In another embodiment of the tattoo machine, the motor assembly further comprises a motor block, a translator, a bearing, and a cam assembly. The motor block further comprises a vertical post. The translator, the bearing, and the cam assembly are stacked on top of the vertical post. The tool can be used to remove and/or disassemble tattoo machine components, such as the tool configured or dimensioned to disassemble or remove the motor assembly, remove the included translator, replace the translator, and reassemble the motor assembly with the new translator, along with the components and parts thereof.

In another embodiment of the tattoo machine, the translator further comprises a translator arm and a translator body, wherein the translator arm is detachably secured to the translator body. The horizontal displacement of the translator arm is adjusted by using the tool to loosen a detachable connection between the translator arm and the translator body, moving the translator arm, and using the tool to tighten the detachable connection between the translator arm and the translator body to detachable secure the translator arm to the translator body.

In another embodiment, the horizontal displacement of the translator arm is adjusted by replacing the included translator with a different translator. In this embodiment, the motor assembly includes a first translator with a first translator arm fixedly secured to the first translator. The first translator arm has a first arm length. The tool is used to disassemble the motor assembly, remove the first translator, replace the first translator with a second translator having a second translator arm with a second arm length, and reassemble the motor assembly with the second translator.

A method of adjusting a tattoo machine comprises disassembling a tattoo machine housing, the tattoo machine housing comprises a motor housing that houses a motor assembly; disassembling the motor housing to access the motor assembly, the motor assembly comprises the translator; adjusting the horizontal displacement of the translator using an tool; reassembling the motor housing; and reassembling the tattoo machine housing.

In an embodiment of the method, the tattoo housing further comprises a grip section detachably connected to the motor housing and a battery housing detachably connected to the motor housing, dissembling the tattoo housing further includes detaching the grip section from the motor housing and detaching the battery housing from the motor housing.

In another embodiment of the method, the motor housing further comprises an exterior motor housing and an interior motor carrier, wherein the interior motor carrier is detachably disposed within the exterior motor housing, wherein the motor assembly is detachably connected to the interior motor carrier. The tool is carried within the interior motor carrier. Disassembling the motor housing further includes separating the exterior motor housing from the interior motor housing.

In another embodiment of the method, the translator further comprises a translator arm and a translator body, wherein the translator arm is detachably secured to the translator body. In this embodiment, adjusting the horizontal displacement of the translator further includes placing a tip of the tool on a detachable connection between the translator arm and the translator body, loosening the detachable connection by rotating the tool in a direction, slidably moving the translator arm to a desired position, tightening the detachable connection by rotating the tool in an opposite direction to secure the translator arm in the desired position.

In another embodiment of the method, the motor assembly includes a first translator with a first translator arm fixedly secured to the translator. The first translator arm has a first arm length. In this embodiment, adjusting the horizontal displacement of the first translator further includes using the tool to disassemble the motor assembly, removing the first translator, replacing the first translator with a second translator having a second translator arm with a second arm length, and using the tool to reassemble the motor assembly with the second translator.

In another embodiment of the method, the motor assembly further comprises a motor block, a translator, a bearing, and a cam assembly. The tool is used to dissemble the motor assembly.

### DESCRIPTION OF DRAWINGS

For a more complete understanding of the present disclosure and its features and advantages, reference is now made to the following description, taken in conjunction with the accompanying drawings, in which:

FIG. 1 is a perspective view of a kit including the tattoo machine and the tool.

FIG. 2 is a perspective view of the tool.

FIG. 3 is a perspective view of an alternative embodiment of the tool.

FIG. 4 is an perspective view of the motor assembly.

FIG. 5 is a cross-sectional view of the motor assembly.

FIG. 6 is a perspective view of the exterior motor housing.

FIG. 7 is a perspective view of the assembled interior motor carrier and motor assembly.

FIG. 8A is a perspective view of an alternative embodiment of the top of the interior motor carrier.

FIG. 8B is a perspective view of an alternative embodiment of the bottom of the interior motor carrier with the tool inserted.

FIG. 8C is a perspective view of an alternative embodiment of the bottom of the interior motor carrier with the tool removed.

FIG. 9A is a perspective view of the top of the interior motor carrier.

FIG. 9B is a perspective view of the bottom of the interior motor carrier with the tool inserted.

FIG. 9C is a perspective view of the bottom of the interior motor carrier with the tool removed.

FIG. 10 is a perspective view of the motor block.

FIG. 11 is a perspective view of the translator.

FIG. 12 is a perspective view of an alternative embodiment of the translator.

FIG. 13A is a perspective view of the cam assembly.

FIG. 13B is a cross-sectional view of the cam assembly.

FIG. 14 is a perspective view of an alternative embodiment of the cam assembly.

FIG. 15 is a perspective view of an alternative embodiment of the cam assembly.

FIG. 16 is a perspective view of an alternative embodiment of the cam assembly.

### DETAILED DESCRIPTION

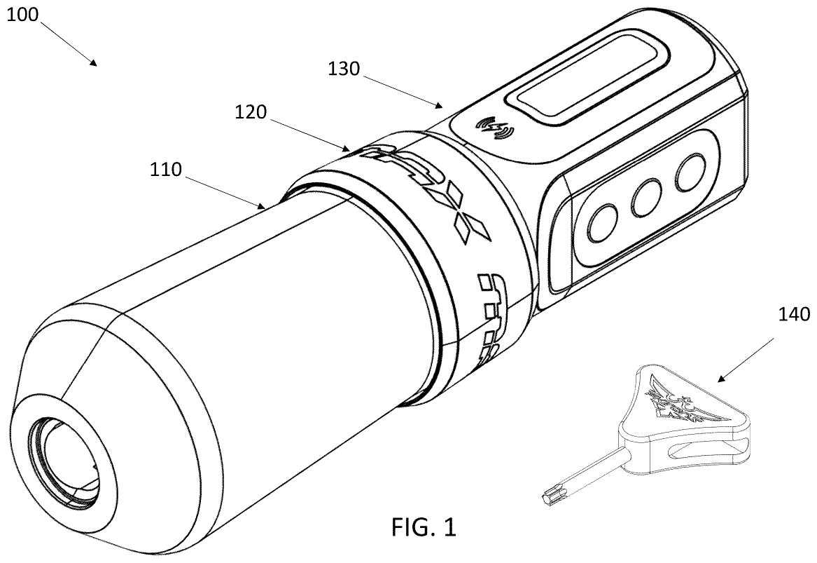

FIG. 1 illustrates a tattoo machine kit including tattoo machine 100 and tool 140 according to some embodiments. The tattoo machine 100 can include a permanent makeup machine, a micropigmentation machine and/or a needling machine. The tattoo machine 100 may include a grip section 110, a motor housing 120, and a battery housing 130. The grip section 110 can be detachably secured to the motor housing 120. The motor housing 120 can be detachably secured to the battery housing 130.

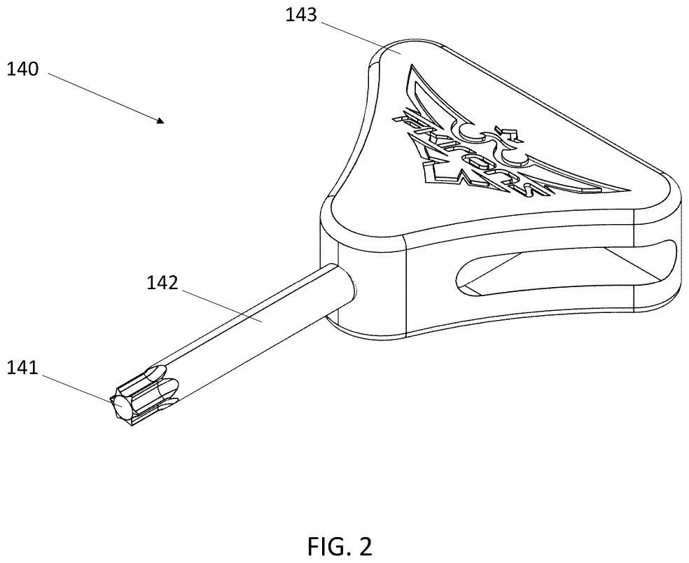

FIG. 2 illustrates a tool 140, such as an adjustment tool 140, according to some embodiments. In other embodiments, the tool 140 can be a tattoo machine tool or a cleaning tool. The tool 140 includes an engagement tip 141, shaft 142, and a handle body 143. The engagement tip 141 may be adapted to engage a variety of fasteners. The drive tip may include a Phillips head, a flat head, a hexagonal head, a pentalobe head or a proprietary head. The handle body 143 may be cylindrical, semicircular, square-shaped, triangular-shaped, pentagonal, hexagonal, octagonal, or a proprietary shape. The handle body 143 may be made out of a rubber material, such as latex or silicone; a plastic material; metal; a combination thereof. The engagement tip 141 and shaft 142 may be made out of metal.

In one embodiment of the tool 140, the engagement tip 141, shaft 142, and handle or tool body 143 are integrally formed with each other. In this embodiment, the tattoo machine kit includes the tattoo machine 100 and a single tool 140 with a single type of engagement tip 141 for engaging a single type of fastener on the tattoo machine 100. In another embodiment, the tattoo machine kit includes tattoo machine 100 and multiple tools 140 with different engagement tips 141 to engage with different types of fasteners on the tattoo machine 100. In another embodiment, the tool 140 includes one or more swappable components that can be attached to, and removable from, handle or tool body 143, such as swapable cleaning tools, swapable tool components. The swappable components can be coupled to the handle or tool body 143 for storage.

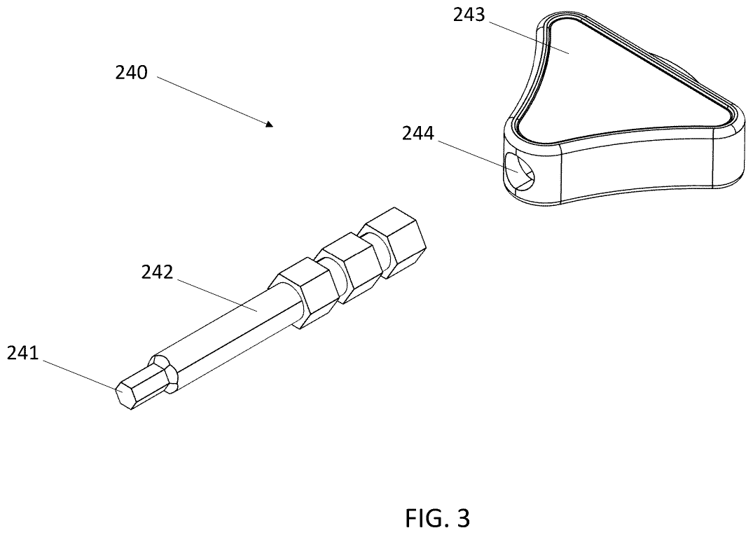

FIG. 3 illustrates an alternative embodiment of the tool 140. In this embodiment, tool 240 has an engagement tip 241, shaft 242, and handle body 243 with a horizontal longitudinal hole 244 on the proximal end of the handle or tool body 243. The engagement tip 241 is integrally formed with the shaft 242. The longitudinal hole 244 receives a portion of the distal end of shaft 242. In this embodiment, the tattoo machine 100 is provided with a single handle body 243 and multiple shafts 242. Each shaft 242 has a different integral engagement tip 241 on the proximal end of the shaft 242 for engaging with different types on fasteners on the tattoo machine 100.

In one embodiment of tool 240, the handle body 242 is magnetic or houses a magnet that magnetically connects integral engagement tip 241 and shaft 242 to the handle or tool body 243. In another embodiment, handle or tool body 243 is magnetic or houses a magnet that magnetically couples to handle or tool body 243 to the machine 100 for storage, which may prevent vibration of tool 240 during use of machine 100.

In another embodiment of tool 240, the distal end of the shaft 242 has a threaded portion that engages with a reciprocal groove within the longitudinal hole 244. In another embodiment of tool 240, the engagement tip 241 and the shaft 242 are detachably secured within the longitudinal hole 244 by a press-fit, where the distal end of the shaft is pushed into the longitudinal hole 244. In this embodiment, the longitudinal hole 244 may be shaped to receive the distal end of the shaft 242. The longitudinal hole 244 may be cylindrical, semicircular, square-shaped, triangular-shaped, pentagonal, hexagonal, octagonal, or a proprietary shape. The distal end of the shaft 242 may correspond to the shape of the longitudinal hole 244.

In another embodiment of the tool 140, the shaft 142 and handle body 143 are integrally formed with one another and the proximal end of the shaft 142 has a socket for detachably securing the engagement tip 141. The engagement tip 141 may be detachably secured to the socket by a push-fit connection, pin-latch connection, or magnetic connection. In this embodiment, the tattoo machine 100 is provided with a single handle body 143 with an integral shaft 142 having a socket at the proximal end of the shaft 142. Multiple different engagement tips 141 are provided to engage with different types on fasteners on the tattoo machine 100.

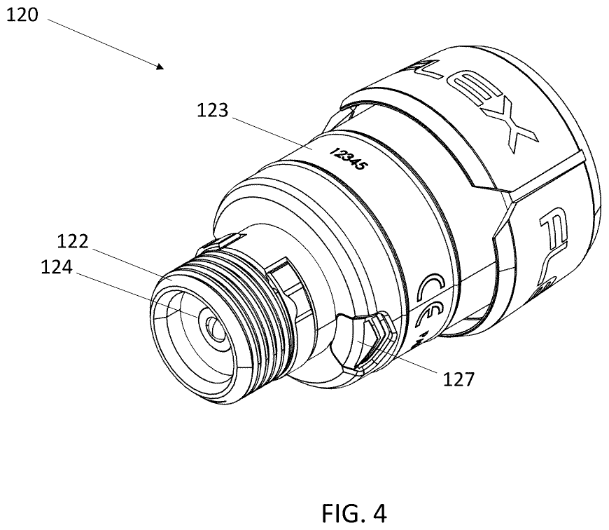

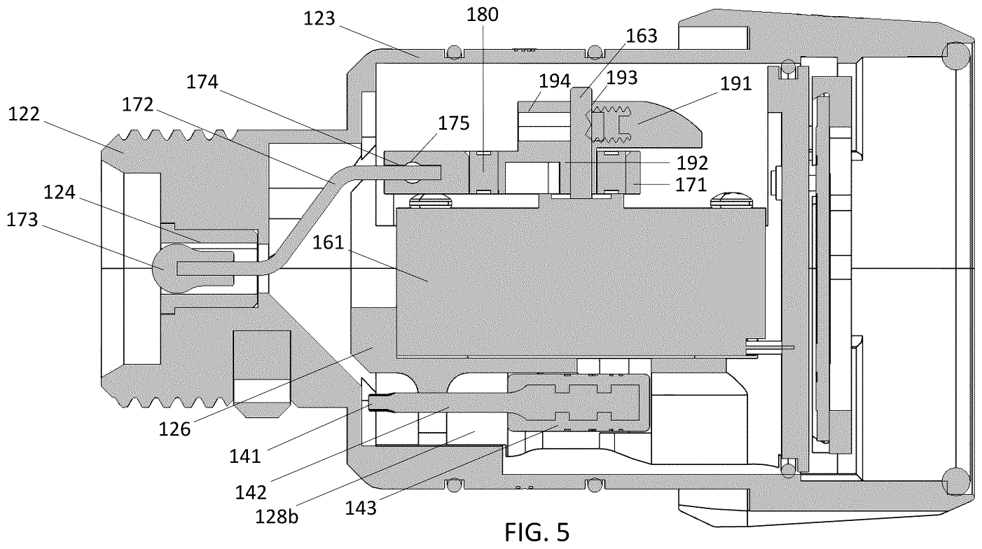

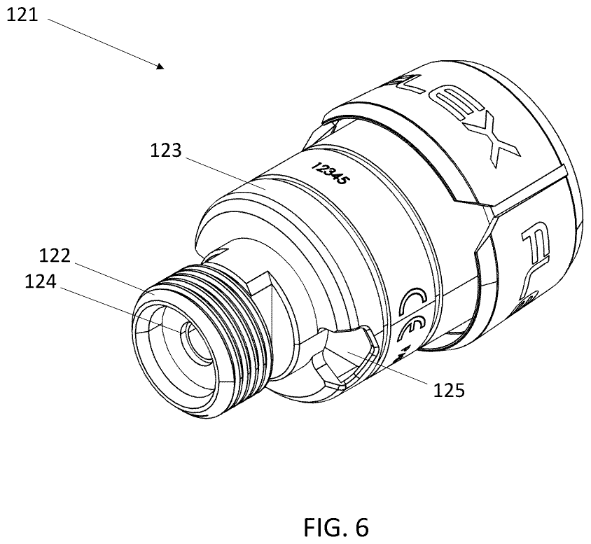

FIGS. 4-6 illustrate a housing 120 according to some embodiments. The motor housing 120 includes an exterior motor housing 121 and an interior motor carrier 126.

The exterior motor housing 121 includes proximal horizontal cylindrical nozzle 122 and a horizontal cylindrical body 123 that are integrally formed with one another. The proximal nozzle 122 may have a plurality of threaded grooves on its exterior surface for detachably securing the motor housing 120 to the grip section 110. The proximal nozzle 122 has an interior channel 124 for receiving a bushing. The cylindrical body 123 has a plurality of notches 125 on its proximal shoulders that engage with the interior motor carrier 126 to detachably secure the interior motor carrier 126 within the exterior motor housing 121. The cylindrical body 123 also has a plurality of circumferential grooves for receiving O-rings to create a fluid tight connection when the motor housing 120 is detachably secured to the grip section 110.

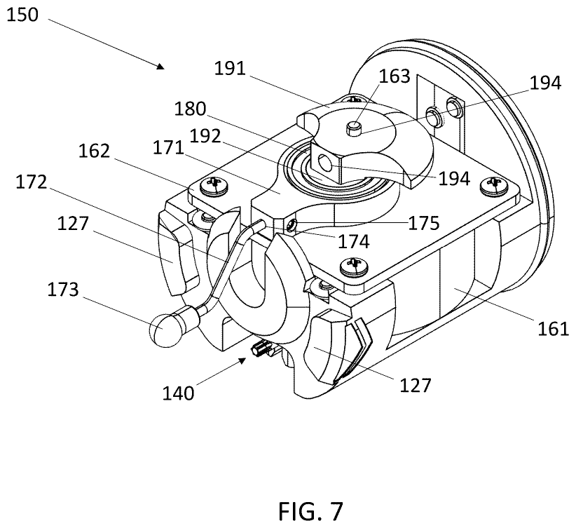

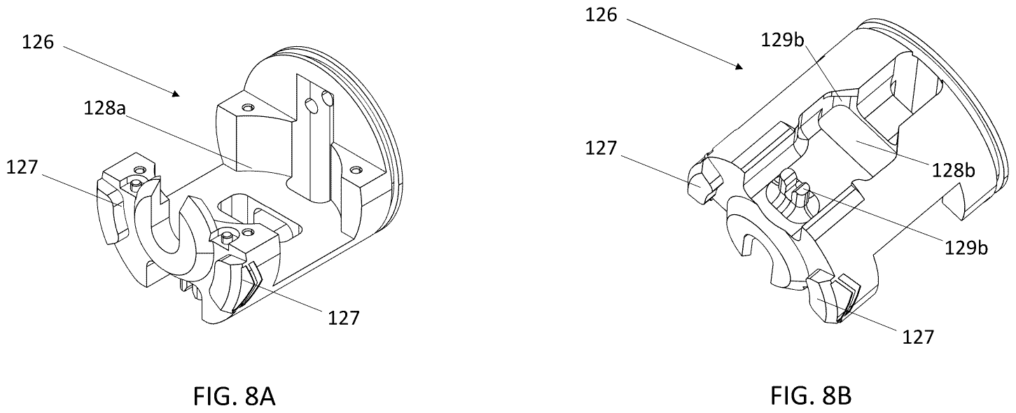

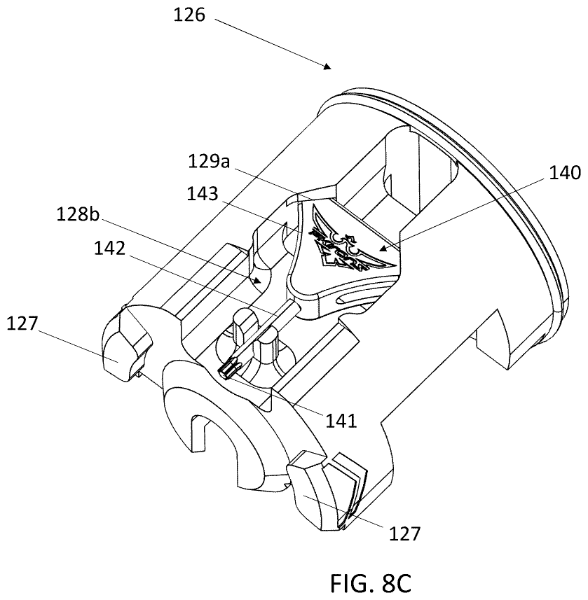

FIGS. 7 and 8A-9C illustrate an interior motor carrier according to some embodiments. As shown in FIGS. 8A-8C, the interior motor carrier 126 has a horizontal cylindrical body with a plurality of compartments 128 and push-buttons 127 on the proximal shoulders of the horizontal cylindrical body 123. The push-buttons 127 engage with the notches 125 on proximal shoulders of the exterior motor housing 121 to detachably secure the interior motor carrier 126 to the exterior motor housing 121. The top of the interior motor carrier 126 has a vertical cylindrical compartment 128a for receiving the motor assembly 150. The portions of the cylindrical body surrounding the cylindrical compartment 128a may contain threaded holes for detachably securing the motor assembly 150 to the interior motor carrier 126. The bottom of the interior motor carrier 126 has or storage compartment 128b for storing the tool 140. The storage compartment 128b may also be used to store spare placement parts for the tattoo machine 100 or motor assembly 150. The tool 140 may detachably secured to the interior motor carrier 126.

FIGS. 8A and 8B illustrate one embodiment of the interior motor carrier 126. In this embodiment, the compartment 128b includes two notches 129a and a clip 129b for detachably securing the tool 140 within the recess. In this embodiment, each notch is located on opposing walls within the compartment 128b and each notch 129a receives a corner of the handle body 143 of the tool 140. The clip 129b is located at the bottom of the compartment 128b and is offset a length from the notches 129a. When the tool 140 is detachably secured within the compartment 128b, the corners of the handle body 143 are first placed within the notches 129a within the compartment 128b. The shaft 142 is then lowered and detachably secured into place by the clip 129b within recess. The handle body 143 of the tool 140 may be rubberized to better secure the tool 140 within the compartment 128b by providing additional friction between the handle body 143 and the notches 129a within the compartment 128b

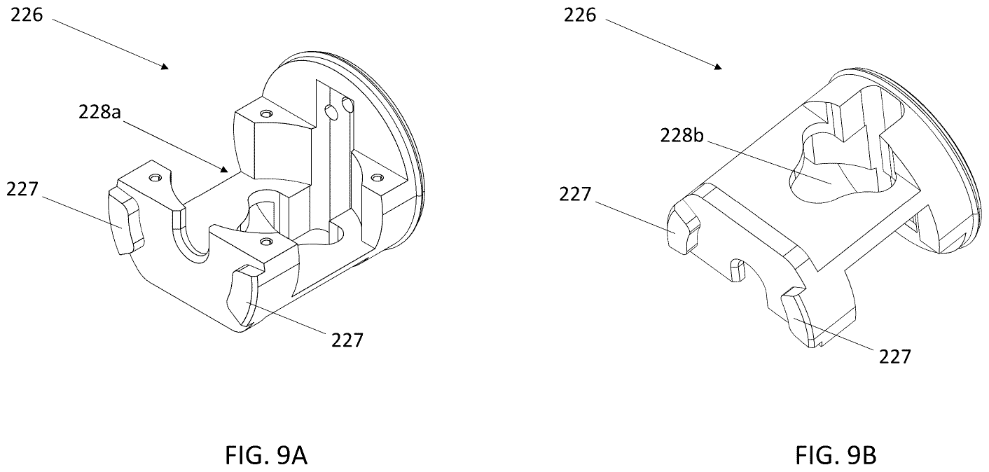

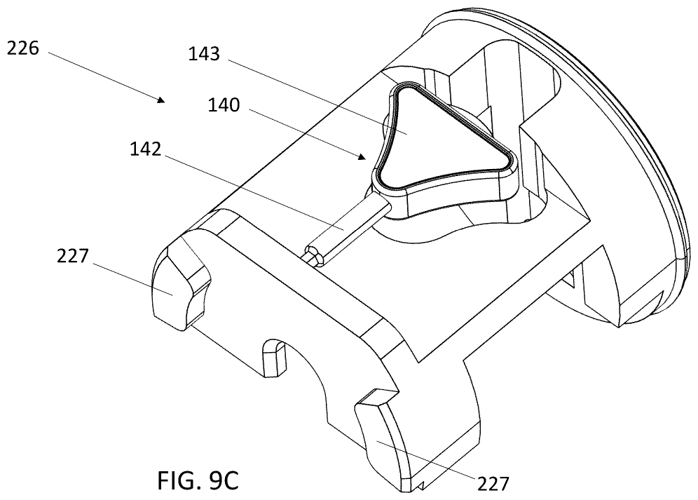

FIGS. 9A-9C illustrate another embodiment of the interior motor carrier 126. In this embodiment the tool 140 may detachably secured to the interior motor carrier 226 by a press-fit. In this embodiment, the recess 228b may be precisely shaped such that the tool 140 is detachably secured to the interior motor carrier 226 by friction between the body of the tool 140 and the interior walls of the recess 228b when the tool 140 is pressed into the recess 228b. The handle body 143 of the tool 140 may be rubberized to better secure the tool 140 within the recess 228b by providing additional friction between the handle body 143 and the interior walls of the recess 228b.

In another embodiment of the interior motor carrier 126, the tool 140 may detachably secured to the interior motor carrier 126 by magnetic coupling. In this embodiment, the body of the tool 140 may be magnetic or the handle body 143 may house a magnet that magnetically that is magnetically attracted to interior motor carrier 126. The compartment 128b may also have a magnet with an opposite pole such that it attracts the magnet in the handle body 143 of the tool 140.

FIG. 7 illustrates a motor assembly 150 according to an embodiment. The motor assembly 150 is detachably secured to the cylindrical compartment 128a on top of the interior motor carrier 126. The motor assembly 150 includes a motor block 160, a translator 170, a bearing 180, and a cam assembly 190.

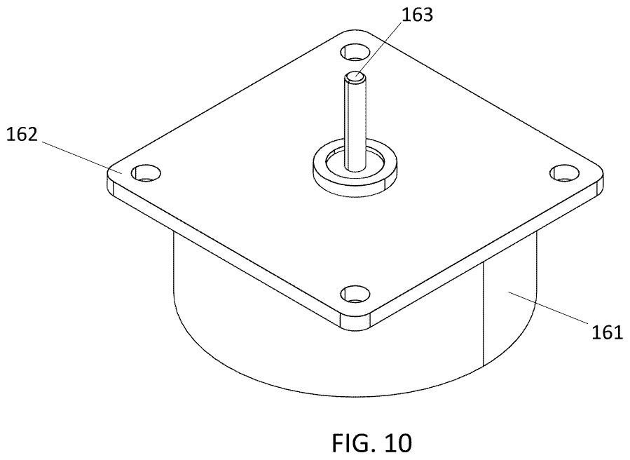

FIG. 10 illustrates a motor block 160 according to an embodiment. The motor block 160 includes a vertical cylindrical body 161, an platform 162, and a perpendicular vertical post 163. The platform 162 is on top of the cylindrical body and the vertical post 163 protrudes from the center of the top of the platform 162. The top of the platform 162 may also include a protruding ring located at the center of the top of the platform 162. The platform 162 may be square-shaped and include a hole for receiving a threaded screw in each corner of the platform 162. The platform 162 may have a width that is approximately equal to the diameter of the cylindrical body 161.

The cylindrical body 161, platform 162, and vertical post 163 may be integrally formed with each other. In another embodiment, the platform 162 and cylindrical body 161 are integrally formed with one another and the vertical post 163 may include a threaded distal portion that detachably connects to a threaded hole at center of the top of the platform 162.

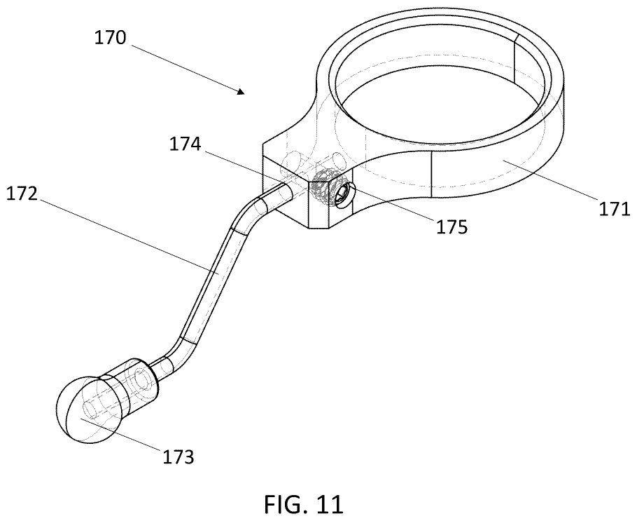

FIG. 11 illustrates a translator 170 according to an embodiment. The translator 170 may include a translator body 171 and a translator arm 172 detachably connected to the translator body 171. In another embodiment of the translator 170, the translator body 171 may be integrally formed or fixedly secured to the translator body 172. The translator body 171 includes a ring with an integrally formed proximal protrusion. The proximal protrusion has a horizontal longitudinal pinhole 174 and a horizontal perpendicular threaded hole 175. The longitudinal pinhole 174 and perpendicular threaded hole 175 are in fluid communication with each other. The longitudinal pinhole 174 receives a distal portion of the translator arm 172. The perpendicular threaded hole 175 receives a threaded insert. The ring has a central cavity for receiving the bearing. The inner diameter of the ring is approximately equal to the outer diameter of the bearing.

When assembling the translator 170, the distal portion of the translator arm 172 is inserted into the longitudinal pinhole 174 in the proximal protrusion of the translator body 171. The threaded insert is then inserted into the perpendicular threaded hole 175 and rotated using the tool 140 until the threaded insert abuts the side of the distal portion of the translator arm 172. The abutment between the threaded insert and the distal portion of the translator arm 172 detachably secures the translator arm 172 to the translator body 171.

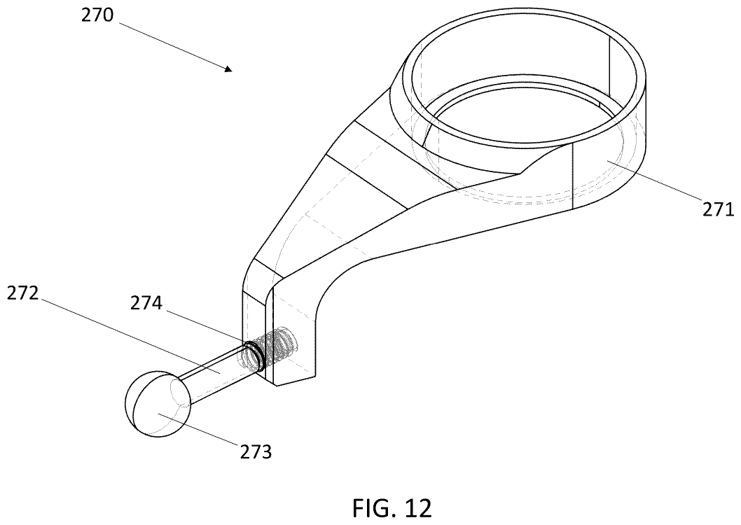

FIG. 12 illustrates an alternative embodiment of the translator 170. In this embodiment, the translator 270 has a translator body 271 and a translator arm 272. The translator body 271 includes a ring with an integrally formed proximal protrusion. The proximal protrusion has a horizontal longitudinal pinhole 274. The distal end of the translator arm 272 may be detachably coupled to the translator body 271 by a threaded connection. In this embodiment, the longitudinal pinhole 274 is threaded and the distal end of the translator arm 272 is threaded with reciprocal threads.

In another embodiment of translator 170, the distal end of the translator arm 172 may be detachably coupled to the translator body 171 by a push-fit connection. In this embodiment, the distal end of the translator arm 172 is detachably connected to the longitudinal pinhole 174 by friction between the exterior surface of the distal end of translator arm 172 and the interior surface of the longitudinal pinhole 174. The longitudinal pinhole 174 is shaped to receive the distal end of the translator arm 172. The inner diameter of the longitudinal pinhole 174 is approximately equal to the outer diameter of the distal end of the translator arm 172. The longitudinal pinhole 174 may be cylindrical, semicircular, square-shaped, triangular-shaped, pentagonal, hexagonal, octagonal, or a proprietary shape. The distal end of the shaft 172 may be correspond to the shape of the longitudinal pinhole 174.

In another embodiment of translator 170, the distal end of the translator arm 172 may be detachably coupled to the translator body 171 by a spring-loaded push-fit connection. In this embodiment, the longitudinal pinhole 174 has a plurality of divots, longitudinally spaced apart from one another along the inner wall of the longitudinal pinhole 174, that are sized to receive a spring loaded ball on the distal end of the translator arm 172. The translator arm 172 is detachably secured to the translator body 171 by frictional engagement between the spring-loaded ball on the distal end of the translator arm 172 and the divots on the inner wall of the longitudinal pinhole 174. In this embodiment, the position of the translator arm 172 can be adjusted by twisting the translator arm 172 such that the spring loaded ball on the distal end of the translator arm 172 is engaged and the spring loaded ball lowers itself into a recess within the translator arm 172. The translator arm 172 can then slide along the longitudinal pinhole 174 until the spring loaded ball engages with another divot on the inner wall of the longitudinal pinhole 174.

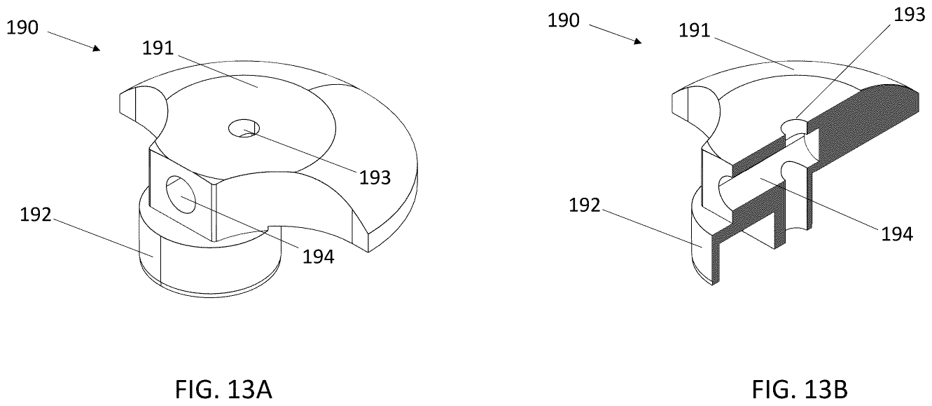

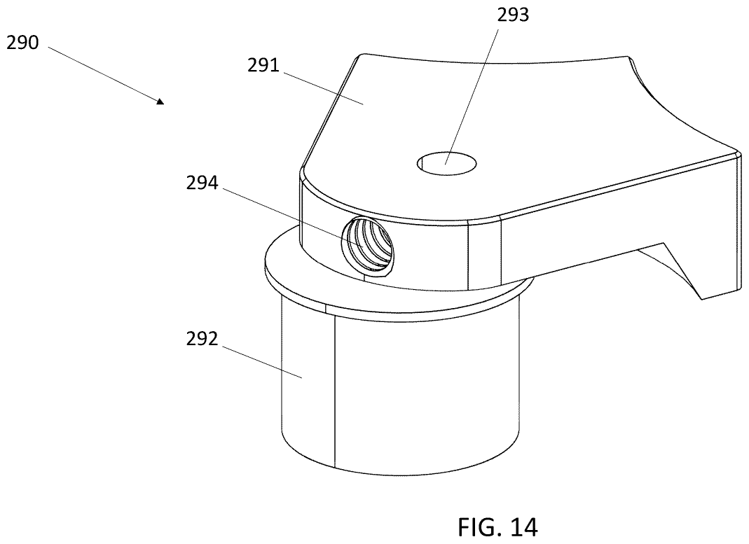

FIG. 13A and 13B illustrates a cam assembly 190 according to an embodiment. The cam assembly 190 has an upper rotor 191 and a lower cylindrical disc 192 that are integrally formed with one another. The lower disc 192 has an outer diameter approximately equal to the inner diameter of the inner central ring of the bearing. The upper rotor 191 has a radius that is larger than the radius of the lower disc 192. The upper rotor 191 may be semicircular, crescent shaped, or shield shaped. The lower disc 192 is longitudinally offset from the center of the upper rotor 191.

The cam assembly 190 further includes a vertical pinhole 193 that runs perpendicularly through both the upper rotor 191 and the lower disc 192. The vertical pinhole 193 starts at the center of the top exterior surface of the upper rotor 191 and ends on the bottom exterior surface of the lower disc 192. The end of the vertical pinhole 193 may be longitudinally offset from the center of the lower disc 192. The vertical pinhole 193 receives the vertical post 163 of the motor block 160. The upper rotor 191 has a horizontal longitudinal threaded hole 194 that receives a threaded insert, which is engaged by an tool 140. The longitudinal threaded hole 194 is in fluid communication with the vertical pinhole 193.

FIG. 14 illustrates an alternative embodiment of cam assembly 190. In this embodiment, cam assembly 290 has an upper rotor 291 that is shield shaped. The base of upper rotor 291 has a horizontal longitudinal threaded hole 294 that receives a threaded insert, which is engaged by an tool 140.

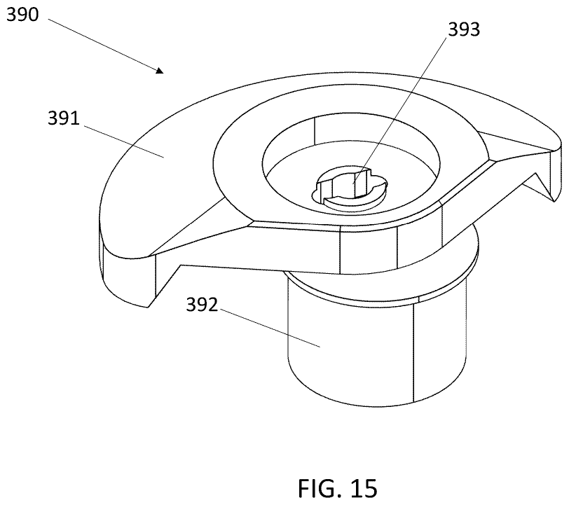

FIG. 15 illustrates an alternative embodiment of cam assembly 190. In this embodiment, cam assembly 390 has an upper rotor 391 and a lower disc 392 that may be separable from each other. In this embodiment, the upper rotor 391 and lower disc 392 may be detachably coupled to one another by the vertical pinhole 393. Lower disc 392 may include a vertically protruding post with a clip that engages with upper rotor 391 to detachably secure upper rotor 391 to lower disc 392. In another embodiment, the upper rotor 191 and lower disc 192 may be detachably coupled to one another by a magnetic connection.

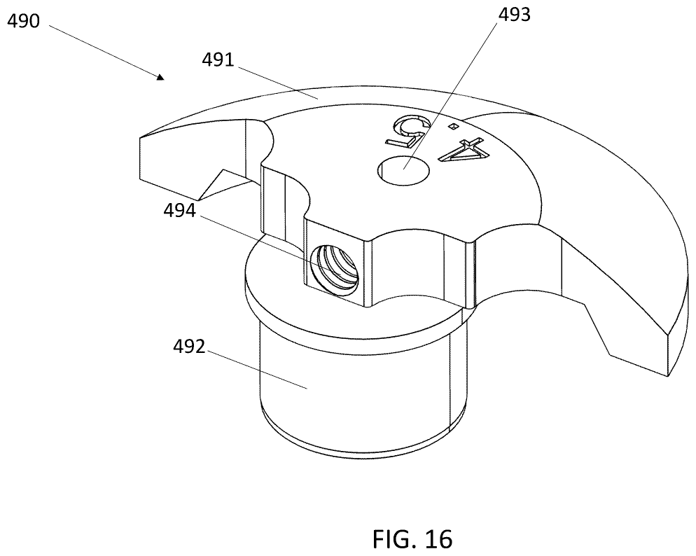

FIG. 16 illustrates an alternative embodiment of cam assembly 190. In this embodiment, cam assembly 490 has an upper rotor 491 and a lower disc 492. The base of upper rotor 491 has a horizontal longitudinal threaded hole 494 that receives a threaded insert, which is engaged by an tool 140.

The motor assembly 150 may be detachably secured to the interior motor carrier 126. The cylindrical body 161 of the motor block 160 is placed with the corresponding cylindrical compartment 128a within the interior motor carrier 126. The motor block 160 is then fastened to the interior motor carrier 126 by placing threaded fasteners through the holes in the corners at the top of the platform 162 of the motor block 160 and rotating the fasteners until the threaded portions of the fasteners engaged with the corresponding threaded holes in the interior motor carrier 126. At this point, the motor block 160 is secured to the interior motor carrier 126 and the vertical post 163 projects perpendicularly upwards from the platform 162 of the motor block 160.

The translator 170, bearing, and cam are then assembled. The bearing is inserted into the central cavity of the translator body 171. The lower disc 192 of the cam assembly 190 is then inserted into the central ring of the bearing. The assembled translator 170, bearing, and cam assembly 190 are then placed onto the motor block 160 by sliding the vertical post 163 on the motor block 160 into an opening on the exterior surface of the bottom exterior surface of the lower disc 192 formed by the vertical pinhole 193.

The vertical post 163 slides down the vertical pinhole 193 until the bottom of the assembled translator 170, bearing, and cam comes to rest on top of the platform 162 of the motor block 160. The threaded insert is then inserted longitudinal threaded hole 194 on integral protrusion on the upper rotor 191 and rotated using the tool 140 until the threaded insert abuts the side of the vertical post 163. The abutment between the threaded insert and the distal portion of the vertical post 163 detachably secures the assembled translator 170, bearing, and cam assembly 190 to the motor block 160.

Once the motor assembly 150 has been detachably secured to the interior motor carrier 126, the interior motor carrier 126 may then be detachably secured to the exterior motor housing 121. The interior motor carrier 126 is inserted into the cavity within the exterior motor housing 121. When the interior motor carrier 126 is inserted into the cavity within the exterior motor housing 121, the push-buttons 127 on the proximal shoulders of the interior motor carrier 126 engage with the corresponding notches 125 on the proximal shoulders of the exterior motor housing 121 to detachably secure the interior motor carrier 126 to the exterior motor housing 121. When the interior motor carrier 126 is inserted into the cavity within the exterior motor housing 121, the proximal tip 173 of the translator 170 protrudes into the bushing in the interior channel in the proximal nozzle 122 of the exterior motor housing 121.

In one embodiment, the tool 140 may be used to adjust the horizontal displacement of the translator 170 and in turn adjusting penetration depth of the needle assembly. In this embodiment, the motor housing 120 must be disassembled to get access to the translator 170.

To disassemble the motor housing 120, a user holds the exterior motor housing 121 in one hand and simultaneously pushes on the push-buttons 127 protruding from the proximal shoulders of the exterior motor housing 121 with their fingertips. This ejects the interior motor carrier 126 from the exterior motor housing 121. The interior motor carrier 126 should slide out from within the inner cavity of the exterior motor housing 121. Once the interior motor carrier 126 is separated from the exterior motor housing 121, the user has access to the motor assembly 150 mounted on top of the interior motor carrier 126 and the tool 140 detachably secured within the compartment 128b at the bottom of the interior motor carrier 126. The user can then detach the tool 140 by pulling up on the shaft 142 of the tool 140, releasing the shaft 142 of the tool 140 from the clip 129b within the recess, and removing the tool 140 from the recess.

In one embodiment, the user can adjust the horizontal displacement of the translator 170 by adjusting the detachable connection between the translator body 170 and the translator arm 172. In this embodiment, the user locates the perpendicular threaded hole 175 on the translator body 171, places the engagement tip 141 of the tool 140 within the perpendicular threaded hole 175, and engages the threaded insert inside the perpendicular threaded hole 175 by turning the tool 140 counter-clockwise. This will retract the threaded insert within the perpendicular threaded hole 175, loosening the translator arm 172 within the horizontal longitudinal pinhole 174 and allowing the translator arm 172 to freely slide within the horizontal longitudinal pinhole 174. The user can then slide the translator arm 172 within the horizontal longitudinal pinhole 174 to the desired distance and secure the translator arm 172 into place by placing the engagement tip 141 of the tool 140 within the perpendicular threaded hole 175, engaging the threaded insert inside the perpendicular threaded hole 175, and turning the tool 140 clockwise until the distal end of the threaded insert abuts against the distal portion of the translator arm 172.

In another embodiment, the user can adjust the horizontal displacement of the translator 170 by using the tool to remove the first translator 170 having a first translator arm 171 with a first arm length and replace it with a second translator 170 having a second translator arm 171 with a second arm length, the second arm length being different from the first arm length. In this embodiment, the user holds the exterior motor housing 121 in the one hand and simultaneously pushes on the push-buttons 127 protruding from the proximal shoulders of the exterior motor housing 121 with their fingertips. This will eject the interior motor carrier 126 from the exterior motor housing 121. The interior motor carrier 126 should slide out from within the inner cavity of the exterior motor housing 121. The user can then hold the interior motor carrier 126 in one hand with the motor assembly 150 facing upward and locate the horizontal longitudinal threaded hole 194 on the upper rotor 191 of the cam. The user can then place the engagement tip 141 of tool 140 into the longitudinal threaded hole 194, engage the threaded insert inside the longitudinal threaded hole 194 with the engagement tip 141 of tool 140, and turn the tool 140 counter-clockwise. This will retract the threaded insert within the longitudinal threaded hole 194 and loosen the abutment between the threaded insert and the vertical post 163. Once the threaded insert has been sufficiently retracted, the user can slidably remove the cam assembly 160, translator 170, and bearing 180 by pulling upward on each component. Each of the cam assembly 160, translator 170, and bearing 180 may also be disassembled into their individual subcomponents. At this point, the user can replace the originally included translator 170 with a different translator 170 having a translator arm 171 with a different arm length.

Once the user has replaced the originally included translator 170 with a different translator 170, the user can then reassemble the motor assembly 150 using the newly selected translator 170. The user can reassemble the motor assembly 150 by placing the cam assembly 160, the new translator 170, and the bearing 180 on the vertical post 163. The user can then place the engagement tip 141 of tool 140 into the longitudinal threaded hole 194 and engage the threaded insert inside the longitudinal threaded hole 194 with the engagement tip 141 of tool 140 and turn the tool 140 clockwise. This will cause the tighten the abutment between the threaded insert and the vertical post 163, securing the cam assembly 160, the new translator 170, and the bearing 180 in place.

Once the user has adjusted the horizontal displacement of the translator 170 to the desired length using either of the methods disclosed above, the user can then reassemble the motor housing 120 by inserting the interior motor carrier 126 into the cavity of the exterior motor housing 121 and sliding the interior motor carrier 126 into the exterior motor housing 121 until the push-buttons 127 on the proximal shoulders of the interior motor carrier 126 engage with the notches on the proximal shoulders of the exterior motor housing 121.

In another embodiment, the tool 140 may be used to disassemble the motor assembly 150 to replace worn out or damaged parts. In this embodiment, disassembly begins by holding the exterior motor housing 121 in the one hand and simultaneously pushing on the push-buttons 127 protruding from the proximal shoulders of the exterior motor housing 121 with their fingertips. This will eject the interior motor carrier 126 from the exterior motor housing 121. The interior motor carrier 126 should slide out from within the inner cavity of the exterior motor housing 121. While holding the interior motor carrier 126 in one hand with the motor assembly 150 facing upward, locate the horizontal longitudinal threaded hole 194 on the upper rotor 191 of the cam. Place the engagement tip 141 of tool 140 into the longitudinal threaded hole 194 and engage the threaded insert inside the longitudinal threaded hole 194 with the engagement tip 141 of tool 140. Turn the tool 140 counter-clockwise. This will retract the threaded insert within the longitudinal threaded hole 194 and loosen the abutment between the threaded insert and the vertical post 163. Once the threaded insert has been sufficiently retracted, the user can slidably remove the cam assembly 160, translator 170, and bearing 180 by pulling upward on each component. Each of the translator 170 and the cam assembly may also be disassembled into their individual subcomponents.

Once the user has identified and replaced the worn out or damaged parts of the motor assembly 150, the user can then reassemble the motor assembly 150. Reassembly begins by placing the cam assembly 160, translator 170, and bearing 180 on the vertical post 163. The user can then place the engagement tip 141 of tool 140 into the longitudinal threaded hole 194 and engage the threaded insert inside the longitudinal threaded hole 194 with the engagement tip 141 of tool 140 and turn the tool 140 clockwise. This will cause the tighten the abutment between the threaded insert and the vertical post 163, securing the cam assembly 160, translator 170, and bearing 180 in place. Once the motor assembly 150 has been reassembled, the motor housing can be reassembled by inserting the interior motor carrier 126 into the cavity of the exterior motor housing 121 and sliding the interior motor carrier 126 into the exterior motor housing 121 until the push-buttons 127 on the proximal shoulders of the interior motor carrier 126 engage with the notches on the proximal shoulders of the exterior motor housing 121.

In other arrangements, the tattoo machine 100 may be disassembled to clean and sterilize various components and/or parts of the tattoo machine 100. Once the tattoo machine 100 and motor assembly 150 are disassembled as described above, the individual components can be cleaned using a variety of cleaning techniques including applying cleaning solutions or using an ultrasonic cleaner. The individual components may be sterilized using medical-grade rubbing alcohol. In another arrangement, the various components of the tattoo machine 100 are autoclavable and may be sterilized using an autoclave. In this arrangement, a user would place the individual components of tattoo machine 100 inside an autoclave and operate the autoclave to sterilize the individual components. Once the individual components have been sterilized, the user can then reassemble the tattoo machine 100 as described above. The ability to disassemble and reassemble the motor assembly 150, motor housing 120, and tattoo machine 100 as described above enables users to more efficiently and effectively clean, maintain, repair, and sterilize the tattoo machine 100, saving the user time and money over the useable life of tattoo machine 100.

The illustrations of arrangements described herein are intended to provide a general understanding of the structure of various embodiments, and they are not intended to serve as a complete description of all the elements and features of apparatus and systems that might make use of the structures described herein. Other arrangements may be utilized and derived therefrom, such that structural and logical substitutions and changes may be made without departing from the scope of this disclosure. Figures are also merely representational and may not be drawn to scale. Certain proportions thereof may be exaggerated, while others may be minimized. Accordingly, the specification and drawings are to be regarded in an illustrative rather than a restrictive sense.

Thus, although specific arrangements have been illustrated and described herein, it should be appreciated that any arrangement calculated to achieve the same purpose may be substituted for the specific arrangement shown. This disclosure is intended to cover any and all adaptations or variations of various embodiments and arrangements. Combinations of the above arrangements, and other arrangements not specifically described herein, will be apparent to those of skill in the art upon reviewing the above description. Therefore, it is intended that the disclosure not be limited to the particular arrangement(s) disclosed as the best mode contemplated for carrying out this application, but that the application will include all embodiments and arrangements falling within the scope of the appended claims.

The foregoing is provided for purposes of illustrating, explaining, and describing embodiments of this application. Modifications and adaptations to these embodiments will be apparent to those skilled in the art and may be made without departing from the scope or spirit of this application. Upon reviewing the aforementioned embodiments, it would be evident to an artisan with ordinary skill in the art that said embodiments can be modified, reduced, or enhanced without departing from the scope and spirit of the claims described below. Although some of the drawings illustrate a number of operations/steps in a particular order, operations which are not order-dependent may be reordered and other operations may be combined or broken out. While some reordering or other groupings are specifically mentioned, others will be apparent to those of ordinary skill in the art and so do not present an exhaustive list of alternatives.

1. A tattoo device, comprising:
a tattoo machine; and
a tool for adjusting the tattoo machine, wherein the tool is detachably housed in a compartment within the tattoo machine.

2. The tattoo device of claim 1, wherein the tattoo machine further comprises:
a tattoo machine housing defining a longitudinal central axis, wherein the tattoo machine housing further comprises:
a grip section;
a motor housing that detachably couples with the grip section;
a battery housing that detachably couples with the motor housing; and
a motor assembly housed within the motor housing.

3. The tattoo device of claim 2, wherein the motor housing further comprises an exterior motor housing and an interior motor carrier.

4. The tattoo device of claim 3, wherein the interior motor carrier is disposed within an inner cavity of the exterior motor housing, wherein the interior motor carrier detachably couples with the exterior motor housing.

5. The tattoo device of claim 4, wherein the tool is carried by the interior motor carrier.

6. The tattoo device of claim 2, wherein the motor assembly further comprises a motor block, a first translator, a bearing, and a cam assembly.

7. The tattoo device of claim 6, where the first translator further comprises a first translator arm, wherein the first translator arm is detachably secured to the first translator.

8. The tattoo device of claim 7, wherein a horizontal displacement of the translator arm is adjusted by using the tool to loosen a detachable connection between the first translator arm and the first translator, moving the first translator arm to a desired position, and using the tool to tighten the detachable connection between the first translator arm and the first translator to detachable secure the first translator arm to the first translator.

9. The tattoo device of claim 8, where the first translator further comprises a first translator arm, wherein the first translator arm is fixed secured to the first translator and has a first arm length.

10. The tattoo device of claim 8, wherein a horizontal displacement of the first translator is adjusted by using the tool to disassemble the motor assembly, removing the first translator, replacing the first translator with a second translator including a second translator arm having a second arm length, and using the tool to reassemble the motor assembly with the second translator.

11. The tattoo device of claim 6, wherein the motor block further comprises a vertical post, wherein the translator, the bearing, and the cam assembly are stacked on top of the vertical post.

12. A method of adjusting a tattoo machine comprising:
instructing a user to:
disassemble a tattoo machine housing, wherein the tattoo machine housing comprises a motor housing that houses a motor assembly and a tool detachably housed in a compartment within the tattoo machine;
disassemble the motor housing to access the motor assembly, wherein the motor assembly comprises a first translator;
adjust the first translator using the tool;
reassemble the motor housing; and
reassemble the tattoo machine housing.

13. The method of claim 12, wherein the tattoo housing further comprises a grip section detachably connected to the motor housing and a battery housing detachably connected to the motor housing, wherein the instructions further include instructing a user to:
detach the grip section from the motor housing, and
detach the battery housing from the motor housing.

14. The method of claim 12, wherein the motor housing further comprises an exterior motor housing and an interior motor carrier, wherein the interior motor carrier is detachably disposed within the exterior motor housing, wherein the motor assembly is detachably connected to the interior motor carrier.

15. The method of claim 12, wherein the tool is carried within the interior motor carrier.

16. The method of claim 12, wherein the instructions further include instructing a user to:
separate the exterior motor housing from the interior motor housing.

17. The method of claim 12, wherein the instructions further include instructing a user to:
place a tip of the tool on a detachable connection between the first translator arm and the first translator,
loosen the detachable connection by rotating the tool in a direction,
slidably move the first translator arm to a desired position, and
tighten the detachable connection by rotating the tool in an opposite direction to secure the first translator arm in the desired position.

18. A tool comprising:
an engagement tip for engaging a fastener on a tattoo machine;
a shaft; and
a handle body, wherein the shaft detachably connects the engagement tip to the handle body;
wherein the tool is configured stored within a compartment within the tattoo machine.

19. The tool of claim 18, wherein the engagement tip can be swapped for a different engagement tip and the shaft can be swapped for a different shaft.
